(19) United States
(12) Patent Application Publication
(10) Pub. No.: US 2026/0253709 A1
(43) Pub. Date: Aug. 27, 2026

(54) COGNITIVE TRAINING PLATFORM

(71) Applicant: NATIONAL UNIVERSITY OF SINGAPORE, Singapore (SG)

(72) Inventors: Dean HO, Singapore (SG); Theodore KEE, Singapore (SG); Christopher Lee ASPLUND, Singapore (SG); Agata BLASIAK, Singapore (SG); Thomas YEO, Singapore (SG)

(73) Assignee: NATIONAL UNIVERSITY OF SINGAPORE, Singapore (SG)

(21) Appl. No.: 19/064,488

(22) Filed: Feb. 26, 2025

Related U.S. Application Data
(63) Continuation of application No. 17/603,115, filed on Apr. 17, 2020, now abandoned.

(30) Foreign Application Priority Data
Apr. 18, 2019 (SG) 10201903518P

(51) Int. Cl. G16H 20/70; G16H 40/67; G16H 50/70

(52) U.S. Cl. CPC G16H 20/70; G16H 40/67; G16H 50/70

(57) ABSTRACT

A cognitive training platform comprises at least one processor that is configured to: obtain a cognitive response function for a user that represents a cognitive state, or change in cognitive state, as a function of one or more stimulus parameters of a stimulus to which the user is exposed; expose the user to a first stimulus that is characterized by respective first values of the one or more stimulus parameters; determine, based on at least one sensor measurement, first cognitive performance values indicative of a response to the first stimulus; determine, using the cognitive response function, respective second values of the one or more stimulus parameters that will result in at least one improved cognitive performance value relative to the first cognitive performance values; and expose the user to a second stimulus that is characterized by the respective second values of the one or more stimulus parameters.

### CROSS-REFERENCE TO RELATED APPLICATIONS

This application is a continuation of U.S. patent application Ser. No. 17/603,115, filed Oct. 12, 2021; which is a 371 of International Application No. PCT/SG2020/050240, filed on Apr. 17, 2020, which claims the benefit of Singaporean patent application number 10201903518P, filed on Apr. 18, 2019. The entire disclosures of the above applications are incorporated herein by reference.

### TECHNICAL FIELD

The present invention relates, in general terms, to a cognitive training platform. The cognitive training platform may have application in digital therapeutics, for example.

### BACKGROUND

Digital therapeutics have emerged as a non-pharmacological alternative for prevention and treatment of cognitive decline and mild dementia, among other conditions. Multiple studies have shown the efficacy of cognitive training delivered in a digital form, to assess dementia state, slow down progression of amnestic mild cognitive impairment, and eventually remediate age-related deficits in cognitive control.

Learning and training regimens in existing cognitive training products are often delivered at a fixed intensity level. This often leads to sub-optimal responses, or even no response at all. Similarly, fixed intensity training can lead to plateaus in learning trajectories and training outcomes. Such training regimens are therefore undesirable for digital therapeutics.

Some existing digital applications for enhancing cognition utilize basic methods of adjusting stimulus intensity, such as task difficulty, based on the user's performance. However, such applications do not take into account the complexity associated with an individual's response to different stimuli at different times.

It would be desirable to overcome or alleviate at least one of the above-described problems, or at least to provide a useful alternative.

### SUMMARY

Disclosed herein is a computer-implemented cognitive training process, comprising:
obtaining a cognitive response function for a user, the cognitive response function representing a cognitive state, or change in cognitive state, as a function of one or more stimulus parameters of a stimulus to which the user is exposed;
exposing the user to a first stimulus, the first stimulus being characterized by respective first values of the one or more stimulus parameters;
determining, based on at least one sensor measurement, one or more first cognitive performance values indicative of a response to the first stimulus;
determining, using the cognitive response function, respective second values of the one or more stimulus parameters that will result in at least one improved cognitive performance value relative to the first cognitive performance value or values; and
exposing the user to a second stimulus, the second stimulus being characterized by the respective second values of the one or more stimulus parameters.

The cognitive response function may be generated by:
exposing the user to a plurality of stimuli, said plurality of stimuli being characterized by time-varying values of said one or more stimulus parameters; and
measuring, by at least one sensor, response data indicative of respective cognitive performance values corresponding to said time-varying values.

In some embodiments, at least two stimulus parameters are varied independently.

The process may comprise fitting said response data to a functional form that is non-linear in the one or more stimulus parameters. For example, the cognitive response function is a quadratic function of the one or more stimulus parameters.

In some embodiments the process comprises obtaining an updated cognitive response function based on the one or more first cognitive performance values; and/or based on one or more second cognitive performance values, the one or more second cognitive performance values indicative of a response to the second stimulus.

The process may comprise monitoring changes in the cognitive response function within a training session and/or across training sessions.

In some embodiments, the process comprises classifying the user into a subpopulation of users; wherein the classification is based on one or more of: the cognitive response function; the updated cognitive response function; and the changes in the cognitive response function.

Advantageously, by classifying the user into a subpopulation, for example based on similarity of their cognitive response function or changes in the cognitive response function to an average cognitive response function of the subpopulation, the behaviour of the subpopulation can be used to make predictions of the user's cognitive performance. For example, a user may have a cognitive response function after a certain number of training sessions that is similar to a subpopulation of users who do not improve in performance after that number of training sessions. That may enable a clinician or other therapist to switch the cognitive training process in favour of another process, as it is likely to be unproductive if continued. In another example, a user may have a cognitive response function that is similar to a subpopulation of users who improve dramatically when particular stimuli are presented to them in future sessions, thereby guiding the clinician to adopt those stimuli.

One or more of said stimuli may be presented via a user interface of a computing device.

In certain embodiments, one of said one or more stimulus parameters is indicative of a training intensity.

One or more of said stimuli may comprise a prompt to provide an input at said computing device. The prompt may be a prompt to provide an input at the user interface of the computing device.

In certain embodiments, the at least one sensor is a sensor of a user input device.

The cognitive response function may also be a function of one or more previously measured cognitive performance values for the user.

Also disclosed herein is a cognitive training platform, comprising:
at least one processor; and
at least one sensor in communication with the at least one processor;
wherein the at least one processor is configured to:
obtain a cognitive response function for a user, the cognitive response function representing a cognitive state, or change in cognitive state, as a function of one or more stimulus parameters of a stimulus to which the user is exposed;
expose the user to a first stimulus, the first stimulus being characterized by respective first values of the one or more stimulus parameters;
determine, based on at least one measurement from the at least one sensor, one or more first cognitive performance values indicative of a response to the first stimulus;
determine, using the cognitive response function, respective second values of the one or more stimulus parameters that will result in at least one improved cognitive performance value relative to the first cognitive performance value or values; and
expose the user to a second stimulus, the second stimulus being characterized by the respective second values of the one or more stimulus parameters.

The at least one processor may be configured to generate the cognitive response function by:
exposing the user to a plurality of stimuli, said plurality of stimuli being characterized by time-varying values of said one or more stimulus parameters; and
measuring, by the at least one sensor, response data indicative of respective cognitive performance values corresponding to said time-varying values.

In certain embodiments, the at least one processor is configured to fit said response data to a functional form that is non-linear in the one or more stimulus parameters. For example, the cognitive response function may advantageously be a quadratic function of the one or more stimulus parameters.

In certain embodiments, the at least one processor is configured to present one or more of said stimuli via a user interface of a computing device.

One of said one or more stimulus parameters may be indicative of a training intensity.

In certain embodiments, one or more of said stimuli comprises a prompt to provide an input at said computing device. The prompt may be a prompt to provide an input at the user interface of the computing device.

At least one sensor of said one or more sensors may be a sensor of a user input device.

In certain embodiments, the cognitive response function is also a function of one or more previously measured cognitive performance values for the user.

Also disclosed herein is a method of obtaining a cognitive response function for a user, the cognitive response function representing a cognitive state, or change in cognitive state, as a function of one or more stimulus parameters of a stimulus to which the user is exposed, the method comprising:
exposing the user to a plurality of stimuli, said plurality of stimuli being characterized by time-varying values of said one or more stimulus parameters;
measuring, by at least one sensor, response data indicative of respective cognitive performance values corresponding to said time-varying values; and
fitting the response data to a functional form that is non-linear in the one or more stimulus parameters, to thereby obtain parameters of the cognitive response function.

The cognitive response function may be a quadratic function of the one or more stimulus parameters.

Further disclosed herein is a non-transitory computer-readable medium having stored thereon instructions for causing at least one processor to perform a cognitive training process according to any preceding paragraph, or a method of obtaining a cognitive response function for a user according to any preceding paragraph.

### BRIEF DESCRIPTION OF THE DRAWINGS

Embodiments of the present invention will now be described, by way of non-limiting example, with reference to the drawings in which:

FIG. 1 is a flow diagram of a cognitive training process according to certain embodiments;

FIG. 2 is a flow diagram of a method of generating a cognitive performance function according to certain embodiments of the invention;

FIG. 3 is a block diagram showing the architecture of a cognitive training platform according to certain embodiments;

FIG. 4 is a block diagram of an example computing device in which certain embodiments may be practised;

FIG. 5 depicts the design of a constant training intensity experiment that does not use the presently disclosed embodiments;

FIG. 6 is an example display of a user interface of a computing system of the cognitive training platform of FIG. 3;

FIG. 7 depicts the design of an experiment in which alternating testing and training blocks were performed;

FIG. 8 shows within-session training effects for the alternating testing and training blocks experiment corresponding to FIG. 7;

FIG. 9 depicts the design of a calibration experiment that uses embodiments of the present invention;

FIG. 10 depicts example cognitive response functions generated by embodiments of the present invention;

FIG. 11 shows cognitive response functions for the first 9 training blocks and the last 9 training blocks in the experimental design of FIG. 7, for a first subject;

FIG. 12 shows cognitive response functions for the first 9 training blocks and the last 9 training blocks in the experimental design of FIG. 7, for a second subject;

FIG. 13 shows cognitive response functions for the first 9 training blocks and the last 9 training blocks in the experimental design of FIG. 7, for a third subject;

FIG. 14 shows performance for three different measures across 7 MATB-II sessions averaged between two volunteers P3 and P6, with P3 and P6 individual scores also shown, and with means across subjects (n=2) plotted in black;

FIG. 15 shows cognitive response functions for volunteer P6 for seven different MATB-II sessions; and

FIG. 16 shows cognitive response functions for volunteer P3 for eight different MATB-II sessions.

### DETAILED DESCRIPTION

Embodiments of the invention relate to a cognitive training process and a cognitive training platform that advantageously make use of user-specific cognitive response profiles, also referred to herein as N-of-1 learning trajectory profiles, to dynamically adjust and thereby optimize a user's response to cognitive training.

Embodiments may identify N-of-1 learning trajectory profiles and learning optimization via a digital interface. By varying the nature the stimulus presented to a user, and measuring the responses to the varying stimulus, it is possible to develop N-of-1 learning trajectory profiles that may actionably mediate training optimization at the single-subject level by dynamically identifying training inputs (for example, the type and/or intensity of the training inputs) that drive the best possible scoring outcome or output relating to cognitive ability and/or state. Accordingly, embodiments of the invention may serve as a powerful optimization platform for digital therapy, student learning, cognitive decline prevention, and other indications.

Advantageously, population-based big data sets are not required by certain embodiments. Synergy prediction between the various inputs is not required in order to globally optimize training for an individual. Empirically recorded or derived measurements or information from the individual can be used to define the individual's profile, used to identify, recommend, and/or be used in a direct feedback based manner to choose a training stimulus that will yield the desired response of the individual.

### Cognitive Training Process 100

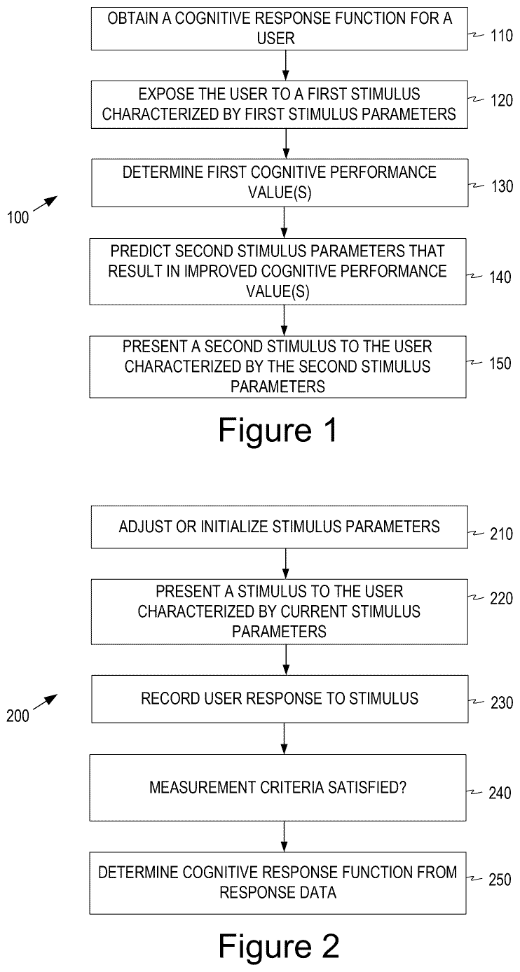

With reference to FIG. 1, an embodiment of a cognitive training process 100 comprises, at block 110, obtaining a cognitive response function for a user. The cognitive training process 100 is implemented at least in part by one or more computer processors. For example, the cognitive training process 100 may be at least partly implemented by a mobile computing device such as a smartphone or tablet, and/or a desktop computing device.

The cognitive response function may be a pre-generated function that is stored on a computer-readable medium and retrieved by the one or more processors as part of the training process 100. Alternatively, the training process 100 may itself generate the cognitive response function, in a manner which will be described below.

The cognitive response function depends on one or more variables and may represent a measurement of a cognitive state or change in cognitive state of the user. The one or more variables include one or more stimulus parameters of a stimulus to which the user is exposed. The one or more variables may also include one or more current or past cognitive state values of the user. That is, in some embodiments, the current cognitive state or change in cognitive state may depend both on the past cognitive state, and the nature and/or intensity of the stimulus to which the user is exposed. In some embodiments, the cognitive response function may depend on one or more variables that characterize the environment of the user, such as ambient temperature, background noise levels, and the like. Accordingly, the stimulus parameters may include both “active” parameters that are controllable, and “passive” parameters that are not controllable but nonetheless measurable such that the impact of their variability on the user's cognitive state (or change in cognitive state) can be determined.

The stimulus may, for example, be presented to the user via a user interface, such as a display of a computing device, another output device such as a speaker or tactile feedback device that is coupled to a computing device, and/or a brain-computer interface. In some embodiments, the stimulus is a prompt to perform one or more tasks, for example a prompt to enter a certain type of input at the user interface, such as tapping or clicking on a target presented on the display, or entering a text response to a question presented on the display. One or more measurements of the response to the stimulus may be made by one or more sensors. For example, the speed and/or accuracy of the response as recorded by an electromechanical sensor of a user input device such as a mouse, keyboard or gesture-based input device may be measured.

In other embodiments, the stimulus may be a visible or audible cue to which the user reacts. One or more sensors may measure a response of the user to the visible or audible cue. For example, a camera may capture one or more images of at least part of the user and determine a cognitive state measurement, for example a cognitive performance measurement (such as reaction speed), based on the one or more images.

At block 120, a first stimulus is presented to the user. The first stimulus is characterized by first stimulus parameters. For example, if the stimulus is a prompt to perform a task, the first stimulus parameters may include an intensity of the task. The intensity may be characterized as low, medium or high, or by a numerical value, for example. In some embodiments, the stimulus may be a prompt to perform multiple different tasks, and the stimulus parameters may be the respective intensities of the tasks, which may be varied together or independently.

At block 130, at least one first cognitive performance value is determined, for example by the computing device that presents the user interface. The first cognitive performance value or values may be determined by capturing data from the one or more sensors, and processing the data to compute one or more numerical values, such as the speed and/or accuracy of the response to the first stimulus.

At block 140, the process 100 determines second stimulus parameters that result in an improved cognitive performance value or values relative to the first cognitive performance value or values. It does so based on the cognitive response function and optionally, the first cognitive performance value(s). For example, process 100 may determine second stimulus parameters that optimize the cognitive response function given the first (i.e., current) cognitive state value. That is, if the cognitive response function is F(x,p) where x is the first (current) cognitive state value and p is the set of parameters characterizing a stimulus, process 100 optimizes F for fixed x to determine second stimulus parameters pₒₚₜᵢₘᵤₘ that result in the optimum value of F. In some embodiments, F may be independent of x, so that all that is required is to optimize a function F(p).

At block 150, a second stimulus is presented to the user, where the second stimulus is characterized by the second stimulus parameters. For example, if the outcome of block 140 is that pₒₚₜᵢₘᵤₘ corresponds to a task intensity of “high”, the process 100 adjusts the user interface (for example) to present a second stimulus at high intensity.

### Calibration Process 200

Turning now to FIG. 2, a process 200 for generating a user-specific cognitive response function is shown. Process 200 may also be referred to as a calibration process.

At block 210, the process 200 begins by initializing respective values of the stimulus parameters.

Next, at block 220, a stimulus is presented to the user, the stimulus being characterized by the initial values of the stimulus parameters. The stimulus is presented by the user interface of the computing device, for example.

At block 230, the user response to the stimulus is recorded. For example, if the stimulus is a prompt to perform a task, one or more sensors (such as electromechanical or optical sensors) measure a user input or other action that is performed in relation to the task, such as a user input made via a mouse, keyboard or other input device. The process 200 may record response data indicative of the speed and/or accuracy of the user input.

At block 240, the process 200 checks whether one or more criteria relating to the measurement have been satisfied. These may include a time criterion (e.g., whether a predetermined time elapsed since process 200 commenced) and/or a requirement for a certain number of measurements.

If the measurement criteria have not been satisfied, process 200 loops back to block 210, where the stimulus parameters are adjusted. The stimulus parameters may be adjusted independently. For example, each stimulus parameter may be adjusted on each iteration, or one or more parameters may be adjusted while the others are maintained at the same level.

Presentation 220 and measurement 230 steps are then repeated, and the process 200 continues until the measurement criterion has, or measurement criteria have, been satisfied.

At block 250, after the one or more measurement criteria have been satisfied, the process 200 determines a cognitive response function from the measured response data. For example, a non-linear function may be fitted to the response data or to values derived therefrom.

In one embodiment, the non-linear function may be a quadratic function of the one or more parameters characterizing the stimulus presented to the user. For example, a healthy and optimized individual's response can be represented as F(S), and a different non-optimized (e.g. mild cognitive impairment/mental decline or healthy baseline) individual by F(S′), where S represents the individual's optimized cognitive/learning/training network mechanisms and S′ the aberrant, sub-optimal, and/or average baseline cognitive/learning/training network mechanisms. The indicator of the individual's cognitive response is the human response of interest that can be measured (e.g. via a digital interface), such as improvement in cognitive performance or function via a quantifiable score (e.g. based on clinically established scoring, game scoring, etc.). The non-optimized individual's response can be parametrized by a parameter C—the manipulation or characteristic (e.g. fatigue) amplitude/level and/or manipulation or characteristic type. Owing to the complexity of these mechanistic networks, explicit forms of these functions—F(S), F(S′), and F(S′,C) —are unknown. F(S′,C) can be expanded about F(S′) to give the following expression:

$$F ( {S}^{′} C ) = F ( {S}^{′} ) + {x}_{0} + \sum {x}_{i} {c}_{i} + \sum {y}_{ii} {c}_{i}^{2} + \sum {z}_{ij} {c}_{i} {c}_{j} + high - order terms \qquad ( 1 )$$

where xᵢ is the individual response coefficient to a factor i (which may be a stimulus parameter or a characteristic of the individual) at amplitude/level cᵢ, and zᵢⱼ is the individual response coefficient to the interaction of manipulation/characteristic i and manipulation/characteristic j at their respective amplitude/level.

Advantageously, the high-order terms (order higher than 2 in the cᵢ) may be dropped from Eq. (1). This enables the introduction of non-linearity into the response, while keeping the number of parameters that must be fitted as low as possible. Because human cognition is thought to respond to inputs in a nonlinear fashion with respect to manipulation i, yᵢᵢ represents a second-order response to the manipulation amplitude/level cᵢ. The values of x₀, xᵢ, yᵢᵢ, and zᵢⱼ can be experimentally determined by calibrating performance outcomes of a specific individual and the manipulation-level inputs (e.g. intensity or difficulty level). Hence, the optimized manipulation-level combination is dynamically personalized to this specific individual, using only their own data. This approach does not require population-level information. Accordingly, the response function can be determined empirically without needing to assume a particular functional form or make any other modelling assumptions.

By moving F(S′) to the left side of Eq. (1) and removing the high-order terms, the following expression is obtained:

$$R ( C ) = F ( {S}^{′} , C ) - F ( {S}^{′} ) = {x}_{0} + \sum {x}_{i} {c}_{i} + \sum {y}_{ii} {c}_{i}^{2} + \sum {z}_{ij} {c}_{i} {c}_{j} \qquad ( 2 )$$

The difference between the two unknown functions F(S′,C) and F(S′) is the overall individual performance response R(C) to the manipulation(s), which can be approximated by a second-order algebraic equation of manipulation levels (stimulus parameters) alone, independent of the specific physiological and/or cognitive mechanisms. Therefore, embodiments provide a platform that is cognitive/learning/training physiological mechanism-independent and disease indication-agnostic. Additionally, because experimental data are used to construct this response surface by calibrating the coefficients, the process 200 is not a model-specific algorithm.

Accordingly, therefore, process 200 may be summarized in a broad sense as including steps of:
exposing the user to a plurality of stimuli, said plurality of stimuli being characterized by time-varying values of said one or more stimulus parameters; and
measuring, by at least one sensor, response data indicative of respective cognitive state values corresponding to said time-varying values.

Once the parameters of cognitive response function R(C) are obtained, they may be used to predict user response to a particular stimulus cᵢ, and to thereby determine a stimulus that will produce an optimized response as discussed above.

Advantageously, in some embodiments, the cognitive response function of a user can be monitored for changes over time. For example, the cognitive response function may change within a training session, and/or between training sessions, thus changing the identified optimised training levels for the desired outcome. To this end, the process 100 and/or the process 200 may comprise determining an updated cognitive response function based on the one or more first cognitive performance values; and/or based on one or more second cognitive performance values, the one or more second cognitive performance values indicative of a response to the second stimulus.

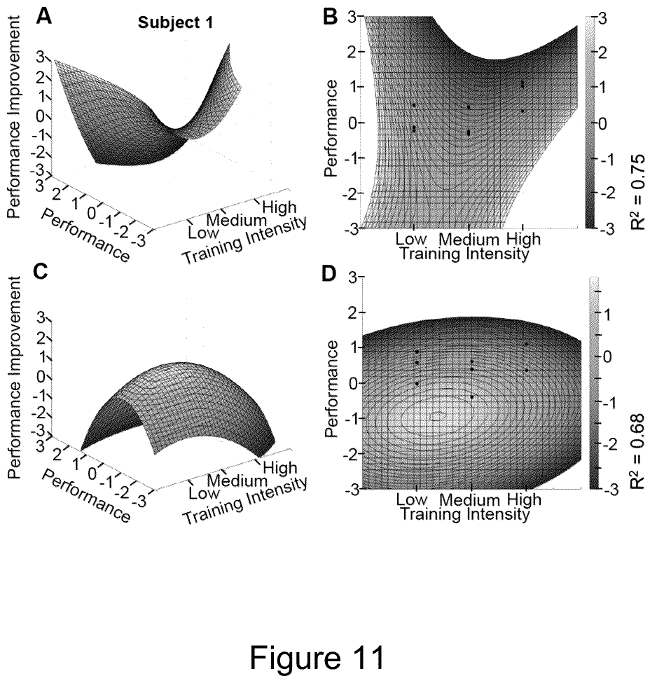

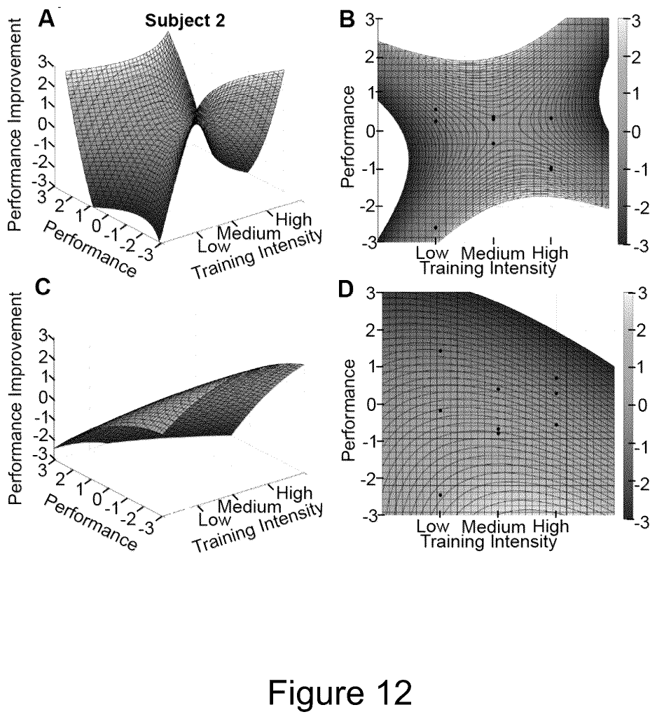

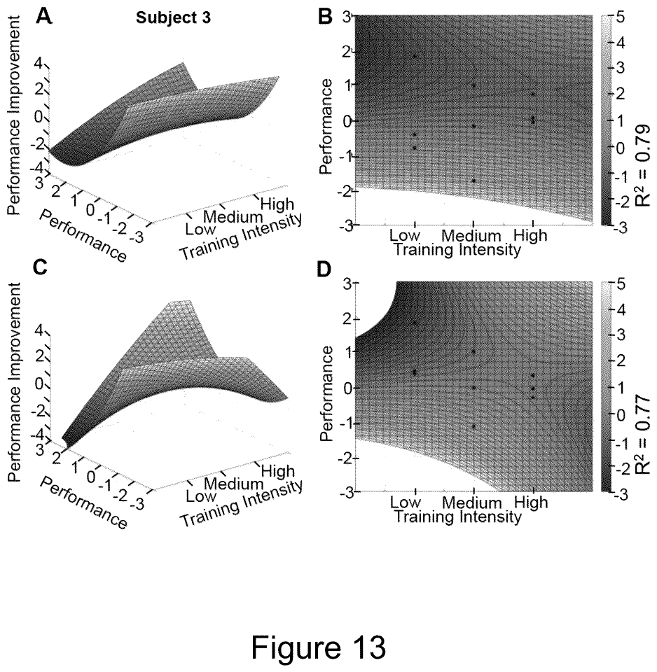

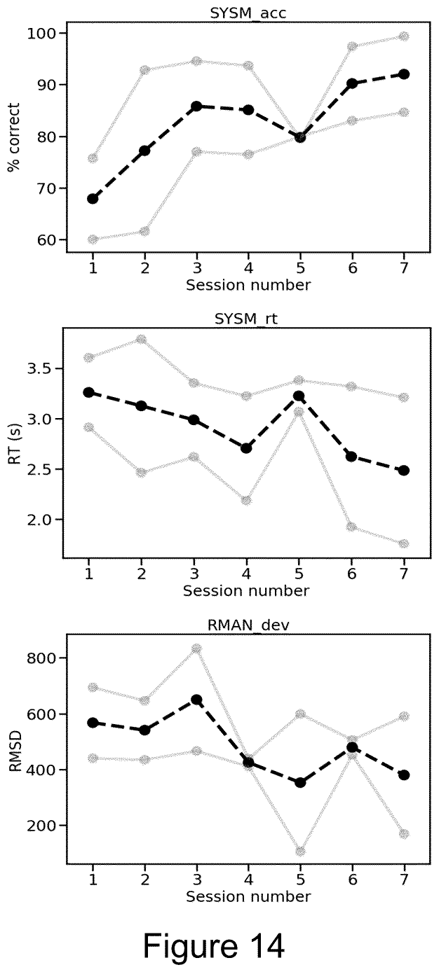

For example, as shown in FIGS. 11-13, the cognitive response function of an individual in the last part of a training session may change relative to that in the first part. This demonstrates that the cognitive response function of the individual is dynamic and that the surface may need to be recalibrated given certain intervals/periods of time to more accurately profile the subject and identify the optimized training intensity (as that may change over time as shown in the two profiles for each individual in FIGS. 11-13). The change in the shape of the profile may be used as an indication of change in the individual's performance (i.e. progression) and potentially as a basis for predicting outcome. Since a minimum number of input/output combinations are needed to calibrate the cognitive response function of the individual, a cutoff can be set, or the input/outputs from earlier in the session or previous sessions can be weighted.

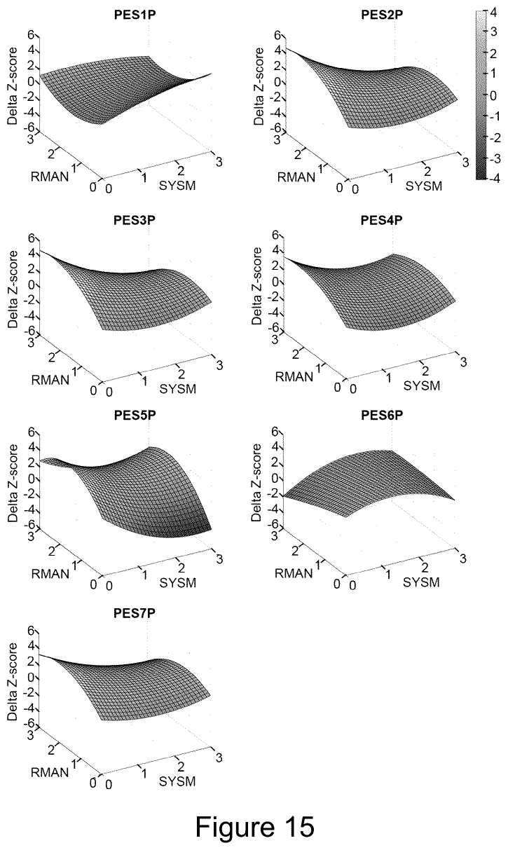

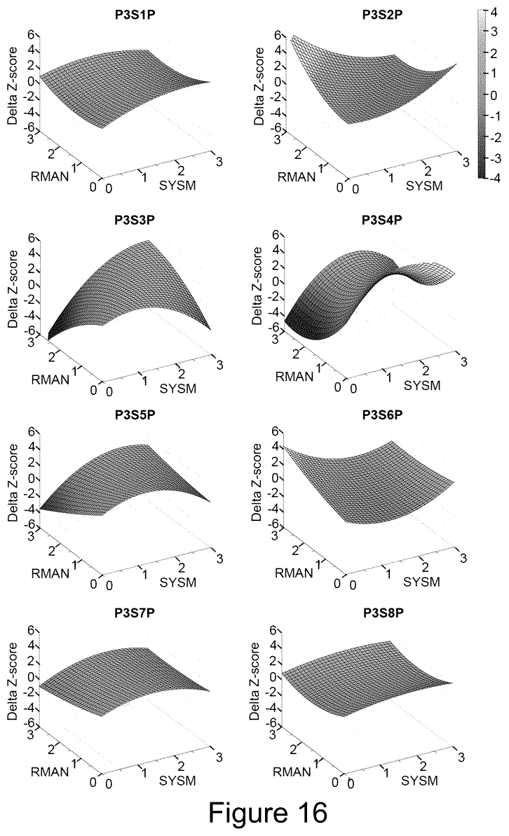

In some embodiments, within-session or session-to-session changes in the behavior of the cognitive response function can be analysed by a comparison with population data. FIGS. 15 and 16 show examples of changes in cognitive response function across sessions, indicating different optimal training intensities for each session as the subject both changed indicating a change in the individuals' performance (i.e. progression) and potentially as a basis for predicting outcome. For example, although population data is not needed to optimise the training regimen for an individual, the changes in the cognitive response function may be used to classify the user by way of comparison with other users who display a similar change profile. The process 100 and/or the process 200 may be performed for a plurality of users who can be grouped into subpopulations by changes in the features of desired outcomes. These identified subpopulations can be used on the level of the individual as a potential predictor of the individual's overall change in the features of desired outcomes.

### Cognitive Training Platform 300

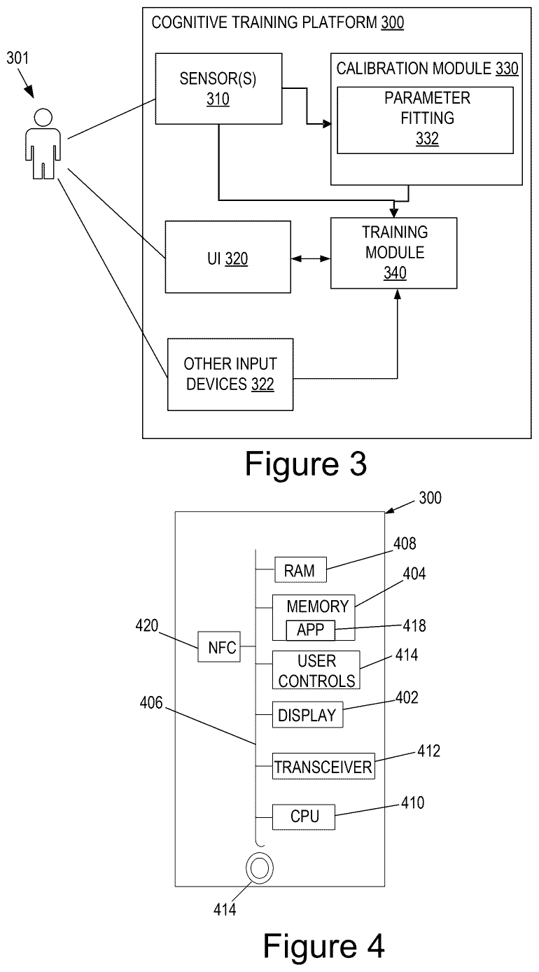

An example cognitive training platform 300, as depicted in FIG. 3, comprises one or more sensors 310, a user interface 320, one or more input devices 322, a calibration module 330 (comprising a parameter fitting sub-module 332), and a training module 340.

The one or more sensors 310 may be electrical, electromechanical, electromagnetic, and/or optical sensors. For example, the sensors may comprise one or more of: a sensor of a user input device such as a keyboard, mouse or stylus; a camera; a microphone; one or more electrodes of a brain-computer interface; or one or more physiological sensors such as a heart rate monitor, a blood pressure sensor, a temperature sensor or a muscle tension sensor.

The user interface 320 may be a display of a computing device, such as a mobile computing device or a desktop computing device. In some embodiments, the user interface 320 may itself include one or more sensors for detecting user input, such as touchscreen sensors (which may be resistive, capacitive, surface acoustic wave, infrared or optical imaging sensors, for example). In addition, one or more other input devices 322 may be provided to detect user input which is detected and analysed by the training module 340.

The calibration module 330 is a hardware and/or software component that is configured to execute the steps of calibration process 200. Calibration module 330 is in communication with sensors 310 and receives data from sensors 310 to determine the response of the user 301 to stimuli that are presented by user interface 320, for example. Calibration module 330 is also in communication with user interface 320, and adjusts the current values of the stimulus parameters to alter the stimulus that is presented by user interface 320 at any given time. Calibration module 330 comprises parameter fitting sub-module 332, that receives the response data indicative of the recorded user responses to the stimuli presented by user interface 320, and fits the parameters of the quadratic form R(C) in Eq. (2) to the response data.

The training module 340 is also a hardware and/or software component that executes the cognitive training process 100. In particular, training module 340 is in communication with the sensors 310, user interface 320 and other input devices 322 to receive data recorded by the sensors 310 and/or other input devices 322, and adjust the stimuli presented by user interface 320 in accordance with the cognitive response function determined by the calibration process 200 of calibration module 330, to thereby optimize the cognitive response of user 301.

The cognitive training platform 300 may have an architecture that is based on the architecture of a desktop or laptop computing device, or a mobile computing device, such as the architecture depicted in FIG. 4 and described below. To this end, the calibration module 330 and training module 340 may be implemented as part of a cognitive training application 418 executed by one or more processors 410 of the mobile computing device (for example) 300.

In other embodiments, the cognitive training platform 300 may comprise a plurality of computing devices, with different components being implemented via different computing devices of the platform. For example, UI 320 and at least some of the sensors 310 may be implemented in a mobile computing device which is operated by the user 301, while calibration module 330 and training module 340 may be implemented in one or more desktop computing devices or servers that are in communication with the mobile computing device.

FIG. 4 is a block diagram showing an exemplary mobile computing device 300 in which embodiments of the invention may be practised. The mobile computer device 300 may be a mobile computer device such as a smart phone, a personal data assistant (PDA), a palm-top computer, or a multimedia Internet enabled cellular telephone. For ease of description, the mobile computer device 300 is described below, by way of non-limiting example, with reference to a mobile device in the form of an iPhone™ manufactured by Apple™, Inc., or one manufactured by LG™, HTC™ and Samsung™, for example.

As shown, the mobile computer device 300 includes the following components in electronic communication via a bus 406:
a display 402;
non-volatile (non-transitory) memory 404;
random access memory (“RAM”) 408;
N processing components 410;
a transceiver component 412 that includes N transceivers;
user controls 414; and
a NFC controller 420.

Although the components depicted in FIG. 4 represent physical components, FIG. 4 is not intended to be a hardware diagram. Thus, many of the components depicted in FIG. 4 may be realized by common constructs or distributed among additional physical components. Moreover, it is certainly contemplated that other existing and yet-to-be developed physical components and architectures may be utilized to implement the functional components described with reference to FIG. 4.

The display 402 generally operates to provide a presentation of content to a user, and may be realized by any of a variety of displays (e.g., CRT, LCD, HDMI, micro-projector and OLED displays).

In general, the non-volatile data storage 404 (also referred to as non-volatile memory) functions to store (e.g., persistently store) data and executable code.

In some embodiments for example, the non-volatile memory 404 includes bootloader code, modem software, operating system code, file system code, and code to facilitate the implementation components, well known to those of ordinary skill in the art, which are not depicted nor described for simplicity.

In many implementations, the non-volatile memory 404 is realized by flash memory (e.g., NAND or ONENAND memory), but it is certainly contemplated that other memory types may be utilized as well. Although it may be possible to execute the code from the non-volatile memory 404, the executable code in the non-volatile memory 404 is typically loaded into RAM 408 and executed by one or more of the N processing components 410.

The N processing components 410 in connection with RAM 408 generally operate to execute the instructions stored in non-volatile memory 404. As one of ordinarily skill in the art will appreciate, the N processing components 410 may include a video processor, modem processor, DSP, graphics processing unit (GPU), and other processing components.

The transceiver component 412 includes N transceiver chains, which may be used for communicating with external devices via wireless networks. Each of the N transceiver chains may represent a transceiver associated with a particular communication scheme. For example, each transceiver may correspond to protocols that are specific to local area networks, cellular networks (e.g., a CDMA network, a GPRS network, a UMTS networks), and other types of communication networks.

The mobile computer device 300 can execute mobile applications. The cognitive training application 418 could be a mobile application, web page application, or computer application. The cognitive training application 418 may be accessed by a computing device such as mobile computer device 12, a desktop computing device or laptop, or a wearable device such as a smartwatch.

It should be recognized that FIG. 4 is merely exemplary and in one or more exemplary embodiments, the functions described herein may be implemented in hardware, software, firmware, or any combination thereof. If implemented in software, the functions may be transmitted or stored as one or more instructions or code encoded on a non-transitory computer-readable medium 404. Non-transitory computer-readable medium 404 includes both computer storage medium and communication medium including any medium that facilitates transfer of a computer program from one place to another. A storage medium may be any available medium that can be accessed by a computer.

### EXAMPLES

### Example 1

A pilot study pertaining to the application of embodiments of the invention towards the derivation of N-of-1 learning trajectory profiles (cognitive response functions) was performed.

We used the Multi-Attribute Test Battery (MATB) platform in this pilot study. Initially developed by NASA and further refined by the US Air Force, the MATB is a flight deck operations simulator that requires the user to perform four tasks concurrently. These tasks include managing fuel tank levels, tracking a target via joystick, adjusting a radio in response to verbal commands, and responding to indicator lights and gauges. Individuals are given instructions for each task, but they must learn effective strategies for task performance and coordination through experience. As such, the difficulty of the tasks could be expected to affect what is learned, though it is challenging—if not impossible—to specify the best difficulty level a priori.

The MATB includes a sophisticated parameterization of task control and user performance, rendering it a potent evaluative tool in several domains. The parameters of the task control may be the parameters p of the first and second stimuli as discussed above.

Foremost, it has been used to characterize subjective and objective measures of mental workload (e.g. scale ratings and EEG signatures) across different levels of task intensity. Individuals perform differently on the MATB even with the same event sequences and control settings, and some of these inter-individual differences have been associated with stable variation in cognitive abilities or personality traits. The MATB has also been used to study improvements in performance with training or experience. Multitasking costs can be reduced or, in some cases, even virtually eliminated without direct instruction. But again, the degree and completeness of such performance improvements varies considerably across individuals. Despite such differences, training regimens typically involve a single difficulty level, and relevant adaptive procedures that could be employed are limited.

Given the previous findings with the platform and its features, the MATB may serve as an ideal candidate for training optimization based on embodiments of the present invention. We tested this notion in three experiments. In the first, we characterized training benefits on each task and interindividual differences in performance, including both baseline and training trajectories. In the second, we tested for training effects on each measure within a single session, and whether task intensity affected performance and improvement rate. In the third, we varied training intensities for each participant, thereby allowing us to attempt the creation of N-of-1 cognitive response functions.

### Experimental Results

Constant Training Intensity over Multiple Sessions

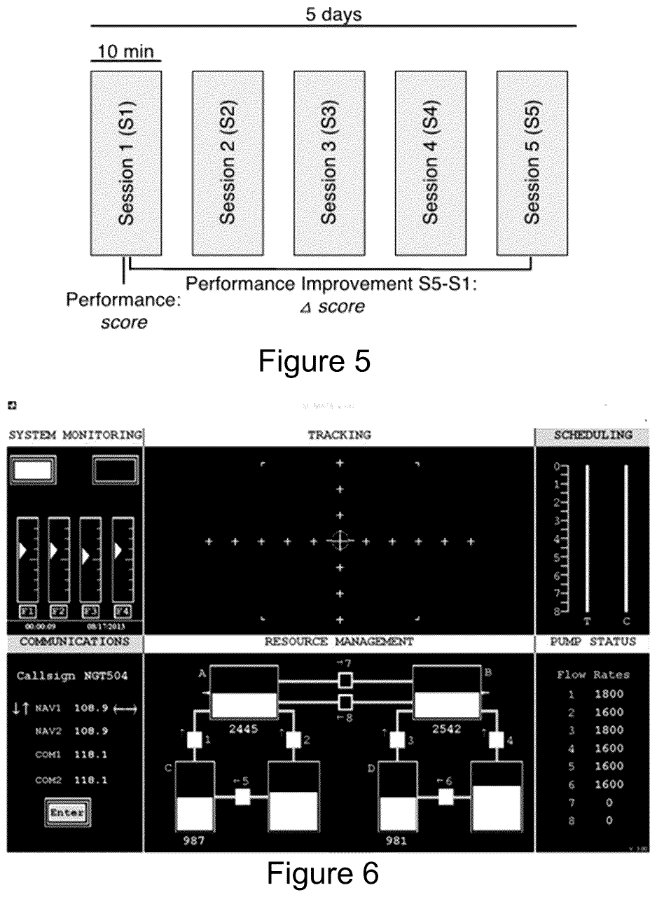

Twenty-eight individuals participated in a five-day training study designed to characterize the variability of individual performance improvement trajectories at a fixed training intensity. A schematic of the training study is shown in FIG. 5. Subjects completed a 10-minute MATB-II session once a day for 5 days (Session 1-Session 5). Subjects' performance scores in each task were recorded for each session. Performance improvement was calculated as the difference (Δ score) between the task scores in the fifth session (S5) and the first session (S1).

During each day's training session, participants completed a 10-minute session of the MATB-II, NASA's current version of the simulator. An example display of MATB-II is shown in FIG. 6. The MATB-II consists of four tasks: Communications (COMM), System Monitoring (SYSM), Resource Management (RMAN), and Tracking (TRCK). Each task was controlled by a script of event timings and settings and collected task-specific key measures as shown in Table 1.

TABLE 1
MATB-II task settings and key measurements. All rates
are given as the average number of events per minute. Each
session lasted for 10 minutes.

| | Event frequencies | |
|---|---|---|
| Task | (per 1 min) | Dependent measures |
| COMM | 1.6 true, 0.8 false | 1. Proportion of correct adjustments |
| | comms | 2. Reaction time for correct adjustments |
| SYSM | 5 gauge deviations, | 1. Proportion of correct responses |
| | 3.5 warning lights | 2. Reaction time for correct responses |
| RMAN | 1.7 pump failures, | Root Mean Square Deviation (RMSD) |
| | continuous fuel flow | between actual and target fuel levels |
| TRCK | Continuous target | Root Mean Square Deviation (RMSD) |
| | movement | between the central crosshair and target |

Across the five days of training, participants improved their performance. Significant training gains were found for five of the six metrics and three of the four tasks, with only Resource Management being too variable for a clear trend to emerge, as shown in Table 2.

TABLE 2
Statistical assessment of training improvements on different components of
NASA's MATB-II. Mean participant performance (n = 28) and changes across sessions
were grouped by measure. Note that all observed changes represent performance
improvements. Changes from the first to last session were assessed using paired t-tests
[t(degrees of freedom), two-tailed], whereas test-retest stability was assessed using
Pearson correlations [r(degrees of freedom), two-tailed]. All data values are reported
as mean ± SD. Statistically significant effects are marked in bold. RT = reaction time.
FL = fuel level.

| | | | Test-retest, | | |
|---|---|---|---|---|---|
| Measure | Session 1 | Session 5 | r(26) | S5-S1 | S5-S1, t(27) |
| COMM (% correct) | 90.3 ± 8.5 | 95.5 ± 4.5 | 0.28 (p = 0.20) | 5.2 ± 8.4 | 3.27 (p = 0.006) |
| COMM (RT in s) | 10.12 ± 0.76 | 9.47 ± 0.44 | 0.58 (p = 0.003) | −0.65 ± 0.62 | −5.55 (p < 0.001) |
| SYSM (% correct) | 73.3 ± 14.3 | 88.2 ± 15.7 | 0.57 (p = 0.005) | 14.9 ± 14.0 | 5.60 (p < 0.001) |
| SYSM (RT in s) | 4.13 ± 0.60 | 2.87 ± 0.51 | 0.58 (p = 0.003) | −1.26 ± 0.51 | −13.0 (p < 0.001) |
| RMAN (FL error) | 593 ± 423 | 578 ± 477 | 0.78 (p < 0.001) | −14 ± 300 | −0.25 (p = 0.80) |
| TRCK (dist. error) | 67.1 ± 16.9 | 47.2 ± 12.3 | 0.66 (p < 0.001) | −19.9 ± 12.8 | −8.23 (p < 0.001) |

There were also significant inter-individual differences in performance on each task. Importantly, these inter-individual differences emerged even though each participant experienced exactly the same event sequence within a given session number. The differences were also relatively stable across sessions (and event sequences), with significant test-retest (Session 1 to Session 5) correlations across participants (Table 2). Nevertheless, training trajectories across the sessions also showed substantial variability that was not fully attributable to either noise or initial performance.

Alternating Training and Testing Blocks within a Single Session

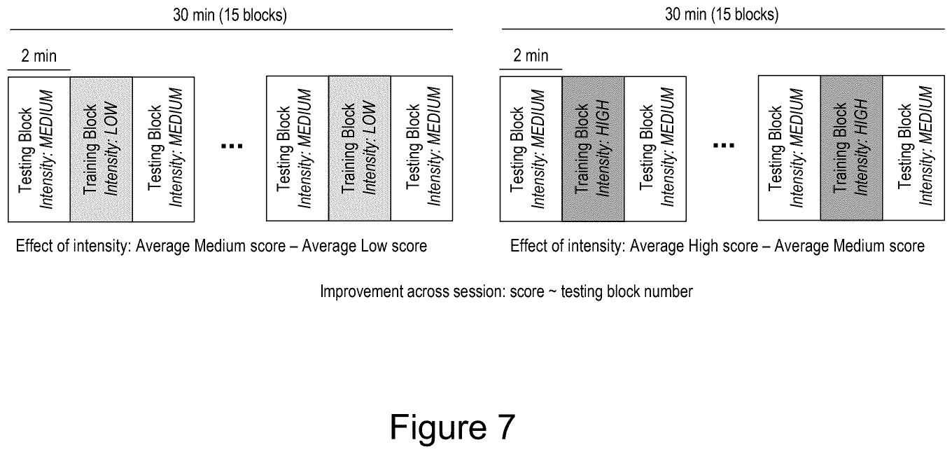

Six individuals completed a pilot study for assessing the feasibility of a design that used alternating testing and training blocks within a single session and determining each task's performance improvement trajectory within such a design. A schematic of the experimental design is shown in FIG. 7. The United States Air Force MATB v3.03 (AF-MATB) was used for this study, as it allows for fine control of each task's intensity (see Table 3). Subjects were randomly allocated to either a high-intensity or low-intensity training group. In a 30-minute session, each subject was presented with alternating training and testing blocks of two minutes each. The transition between blocks was seamless, and blocks differed only in intensity and how they were intended to be used for analysis. Testing blocks were set at medium intensity and were used for measuring performance. Training blocks were set to either low or high intensity, with settings chosen to produce markedly different experiences of difficulty while keeping the tasks feasible for most participants. In our experimental setup, the aim of these blocks was to improve performance.

Performance in each block was sensitive to training intensity. Comparisons between average performance on training blocks and average performance on testing blocks by subject revealed that performance was significantly worse on the TRCK task and COMM reaction time metric, and marginally worse on the SYSM reaction time metric, during higher-intensity blocks (Table 4).

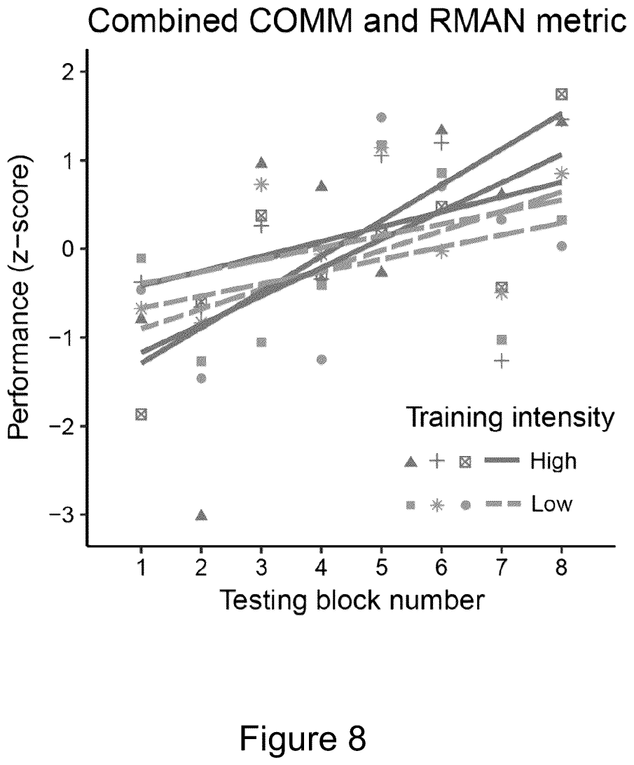

Performance also improved across time within even the single MATB session (Table 5). As measured by each subject's performance change slope across the eight testing blocks, significant improvements were found for the COMM and RMAN tasks, whereas the other two tasks remained statistically unchanged. We therefore combined the metrics for the tasks showing improvement, calculating a sum of each measure's z-scores across blocks to produce an overall performance metric sensitive to training gains (FIG. 8). This combined metric was used for the creation of individual profiles (cognitive response functions) in the final experiment described below.

TABLE 3
AF-MATB task settings for each training intensity (low, high) and
testing intensity (medium). All rates are given as the average
number of events per minute. (Each block was 2 minutes in
duration.) “Comm” stands for “communication event”.

| | Low | Medium | High |
|---|---|---|---|
| COMM | 1 true, 4 false | 2.5 true, 2.5 false | 4 true, 1 false |
| | comms | comms | comm |
| SYSM | 2.5 gauge | 5 gauge deviations | 7.5 gauge |
| | deviations | | deviations |
| RMAN | 1 failure, 1 | 2 failures, 2 | 4 failures, 4 |
| | shut-off | shut-offs | shut-offs |
| TRCK | Low difficulty | Moderate difficulty | High difficulty |

TABLE 4
Results for effects of intensity on performance. Mean participant
performance across participants (n = 6) for each condition.
One-sample t-tests [t(degrees of freedom)] for each
measure assessed whether mean block intensity differences
(Medium-Low or High-Medium) across subjects were significantly
greater than 0 (one-tailed test). All data values are
reported as mean ± SD. Statistically significant
effects are marked in bold. RT = reaction time. FL = fuel level.

| | Low | Medium | High | |
|---|---|---|---|---|
| | intensity | intensity | intensity | Intensity |
| Measure | (Training) | (Testing) | (Training) | effects, t(5) |
| COMM | 87.4 ± 21.7 | 84.3 ± 20.2 | 74.4 ± 21.8 | −2.49 |
| (% correct) | | | | (p = 0.055) |
| COMM | 8.59 ± 1.65 | 9.23 ± 1.33 | 9.37 ± 1.11 | 1.72 |
| (RT in s) | | | | (p = 0.15) |
| SYSM | 31.4 ± 39.2 | 35.6 ± 27.8 | 36.5 ± 14.9 | −2.36 |
| (% correct) | | | | (p = 0.065) |
| SYSM | 2.69 ± 2.46 | 3.73 ± 2.10 | 4.80 ± 1.63 | −0.50 |
| (RT in s) | | | | (p = 0.64) |
| RMAN | 1010 ± 427 | 1190 ± 577 | 1330 ± 652 | −0.33 |
| (FL error) | | | | (p = 0.75) |
| TRCK | 83.0 ± 28.0 | 110 ± 28.1 | 128 ± 29.8 | 5.33 |
| (dist. error) | | | | (p = 0.003) |

TABLE 5
Results for improvement across the session based on testing block
performance. Average performances and changes by testing block
across participants (n = 6) were grouped by measure. Changes across
testing blocks are represented by slopes from linear regressions. One-
sample t-tests [t(degrees of freedom)] assessed whether the slopes for
each measure across participants were greater than 0 (one-tailed
test), with significant results marked in bold. RT = reaction time.
FL = fuel level. All data are demonstrated as mean ± SD.

| | Average | Change | Change by block, |
|---|---|---|---|
| Measure | performance | by block | t(5) |
| COMM | 84.3 ± 20.2 | 2.60 ± 3.08 | 2.07 (p = 0.093) |
| (% correct) | | | |
| COMM | 9.23 ± 1.33 | −0.280 ± 0.130 | −5.26 (p = 0.003) |
| (RT in s) | | | |
| SYSM | 35.6 ± 27.8 | 1.30 ± 2.42 | 1.31 (p = 0.25) |
| (% correct) | | | |
| SYSM | 3.73 ± 2.10 | 0.066 ± 0.165 | 0.97 (p = 0.37) |
| (RT in s) | | | |
| RMAN | 1190 ± 577 | −67.0 ± 59.9 | −2.74 (p = 0.041) |
| (FL error) | | | |
| TRCK | 110 ± 28.1 | −0.50 ± 3.05 | −0.40 (p = 0.71) |
| (dist. error) | | | |

### Modulated Training Intensity

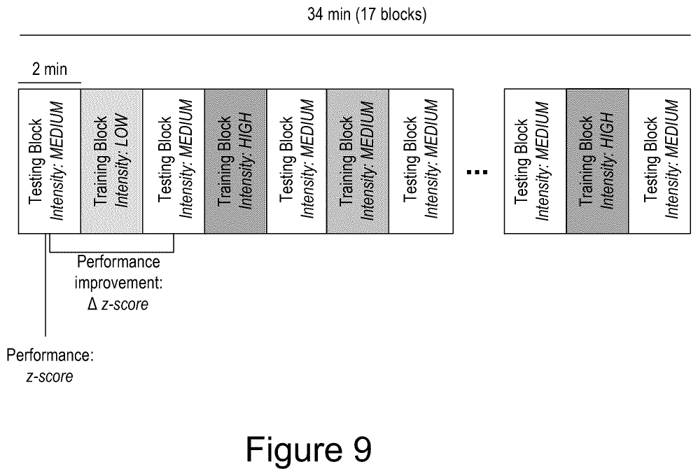

Three individuals successfully completed a pilot study to demonstrate the ability to identify N-of-1 optimization of performance improvement in a single session. Two additional individuals stopped performing one or more component tasks; their cases are discussed below, but they were not used for creating individualized profiles. The AF-MATB session was composed of alternating two-minute testing blocks set at medium intensity collecting performance score, and two-minute training blocks set at low, medium, or high intensity, with training intensity defined per task (Table 3). Performance improvement was defined as the difference in performance during testing blocks before and after the intervening training block (FIG. 9).

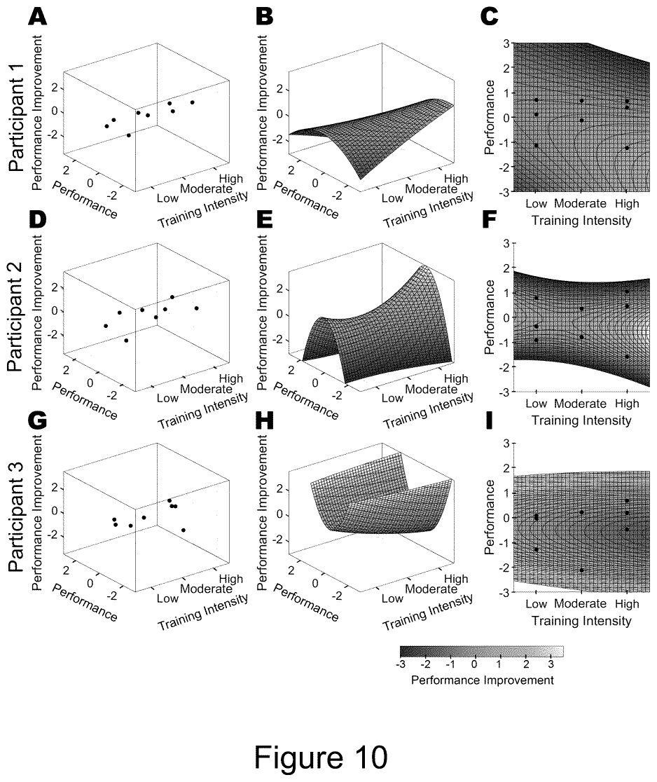

Individualized profiles for subjects 1-3 were constructed and found to represent the unique relationship between training intensity and performance from the training blocks with performance improvement from the collected AF-MATB data (FIG. 10). Importantly, each subject's profile is based only on their own prospectively obtained data. This profile could then be used as a map to dynamically guide training intensity based upon performance. Subject 1 had a performance range of −1.24 to 0.70 (FIG. 10A), and subject 1's profile yielded a 0.66 R-squared value correlation and 0.81 fitting correlation (FIG. 10B,C). The profile demonstrates an elongated convex surface, which indicates dependency between performance and training intensity (FIG. 11B).

Subject 2 had a performance range of −1.59 to 1.04 (FIG. 10D), and subject 2's profile yielded a 0.74 R-squared value correlation and 0.86 fitting correlation (FIG. 10E,F). The profile demonstrates a saddle-like transition between two convex portions of the surface (FIG. 10E). Subject 3 had a performance range of −2.13 to 0.66 (FIG. 10G), and subject 3's profile yielded a 0.92 R-squared value and 0.96 fitting correlation (FIG. 10H,I). The profile demonstrates a concave surface (FIG. 10H). Each subject's profile is unique and provides a pathway towards N-of-1 training optimization in a sustained manner based on continued or repeated testing (FIG. 10).

### Discussion

Based on the performance profiles during both multiple and single training sessions, the MATB demonstrated its potential utility as a platform for performance optimization based on embodiments of the present invention. Across five sessions of training, performance improvements were substantial for almost all metrics and MATB tasks. Furthermore, even without modulating task difficulty, baseline performance and improvement trajectories varied greatly across individuals. These same features were observed even during a single session with modulated training intensity. In addition, participants were sensitive to these training intensity manipulations, with performance during higher-intensity blocks generally poorer compared to lower-intensity blocks. Modulation of training intensity may therefore be similar to the dose modulations to which embodiments of the present platform has now been extensively and successfully applied.

The profiles from individual subjects further demonstrate the potential of embodiments of the present invention for optimizing behavioral performance and its rate of improvement. The individual surfaces varied in overall shape, ranging from convex to saddle-like. Specifically, the convex behavior of the profile for subject 1 (FIG. 10B) is relevant to when the subject initially began training on MATB, indicated by the low and negative performance, a high training intensity would have achieved the highest performance improvement. As the subject's performance improved to a moderately positive performance, associated with the flatter portion of the subject's profile, all three training intensities would yield similar performance improvement (FIG. 10C). Saddle-like behavior of the profile for subject 2 (FIG. 10E) indicates that the highest performance improvement is yielded when the performance scoring around zero value is matched with high intensity training. Interestingly, low intensity training yields a higher improvement when compared to medium intensity training (FIG. 10F). The concave behavior of the profile for subject 3 (FIG. 10H) indicates that at the performance scoring around zero value the highest performance improvement is yielded when the subject is given the low intensity training (FIG. 10I). This interpretation is in contrast to that drawn based on the saddle like behavior of the profile for subject 2, stressing the potential utility of the present embodiments as a tool to guide training intensity at the individual level in order to obtain the highest performance improvement. As each subject's performance improves, the subject's profile will shift and evolve allowing dynamic training intensity modulation.

Importantly, training intensity is also not expected to have a monotonic effect on training improvements. Difficulty settings that are too high may result in individuals giving up on one or more tasks, whereas settings that are too easy may result in little and inefficient learning. Indeed, out of the five subjects recruited for the modulated training intensity experiment, one subject resigned from performing the resource management task when training intensity increased to the high level, and one subject ceased to perform the communication task. Under other circumstances, such difficulty settings may be beneficial. For example, easier settings may enable individuals to focus on improving each task's performance to their overall benefit, whereas difficult settings may be needed to detect and address “latent bottlenecks” in multitasking. These latent bottlenecks induce coordination problems or other costs that are only revealed in challenging circumstances. As such, further performance gains could be found by stretching the training intensity space, a key feature of cognitive training optimization using the presently described embodiments. As noted above, the most useful training intensity will vary across the course of training and across individuals. Such differences will be due in part to the specific difficulties an individual has with a given task, as well as stable inter-individual differences in general cognitive or motivational capacities.

Individuals in multi-tasking situations often trade-off performance across tasks, improving on one by sacrificing effort on another. For example, the two participants who each stopped performing a task in the modulated training intensity experiment potentially could not cope with the demands of the high-intensity training blocks. Unfortunately, their solution effectively halted learning of the dropped task and coordination across the full set of tasks. Less dramatically, the tasks affected by training intensity modulation were often not the tasks that showed training gains. Individuals may trade off performance as a deliberate strategy, or such effects may emerge as a byproduct of the training procedure. Regardless of the cause, the presently disclosed embodiments may be useful for optimizing desired regimen compliance.

### Experimental Section

Participants. The study was conducted according to a protocol approved by NUS Institutional Review Board (S-17-180) and listed on Clinicaltrials.gov (identifier NCT03832101). In total, 41 individuals were recruited, gave informed consent, and participated in MATB simulator experiments. Participants were required to be fluent in English and to have no history of perceptual or memory deficits. No participants had prior experience with the MATB or a similar platform.

Apparatus. The MATB is a flight deck simulator with versions developed by the National Aeronautics and Space Administration (NASA) and United States Air Force (USAF). The two MATB versions, NASA's MATB-II (v2.0), and the USAF's AF-MATB (v3.03) use highly similar displays and interfaces (FIG. 6). All experimental sessions were run on a PC laptop running Windows 8, with sounds played through Creative headphones and input from a Thrustmaster VG T16000M FCS joystick and keyboard. All pointing was done with a mouse for MATB-II sessions, while either a mouse or trackpad was used for AF-MATB sessions.

Tasks and Measures. The MATB consists of four primary tasks: Communications (COMM), System Monitoring (SYSM), Resource Management (RMAN), and Tracking (TRCK). All four tasks were used in each experiment in a similar way. In the Communications (COMM) task, participants acted upon messages preceded by their call sign (true comms), while ignoring other messages (false comms). True comms required the participant to select the radio and adjust its frequency using the mouse, trackpad, and/or keyboard; the speed and accuracy of these responses were the dependent measures. System Monitoring (SYSM) consisted of two subtasks, lights and gauges. For the former, participants needed to click on a green indicator light if it turned off and on a red indicator light if it turned on (FIG. 6). For the latter, participants needed to click on any gauge whose indicator drifted more than two marks away from the midpoint. Due to a task window problem, only the gauge subtask was performed and analyzed for experiments that used the AF-MATB. Response accuracy and speed were the dependent measures for each subtask. The Resource Management (RMAN) task required participants to continually manage a set of pumps, switching each on and off in order to maintain fuel levels near 2500 in the two main fuel tanks. Pump failures (pump could not be used) and pump shut-offs (pump could be reactivated with a click) also occurred. The Tracking (TRCK) task required the participant to continuously track a moving target via joystick. For RMAN and TRCK, the dependent measure was root mean square deviation (RMSD) from the target fuel level or the target position relative to the crosshair.

Each task was controlled by a script of event timings and other settings (e.g. tracking difficulty). Experiment-specific settings for each task and condition are listed in Tables 1 and 3.

Procedure. Participants were seated approximately 60 cm from the screen for each session. During the first (or only) session, participants were instructed on each task and allowed to experience each task in isolation, prior to the real experimental session. On subsequent days, no additional familiarization was provided. Specific procedural and analytical aspects of the three experiments follow below.

Constant Training Intensity over Multiple Sessions Experiment. Twenty-eight individuals (12 males, 27 right-hand dominant, mean age of 23.0, age range of 19-30) were recruited for a five-day training study. On each day, participants completed several cognitive tasks for up to 90 minutes total, including a 10-minute session of the MATB-II. Identical settings and event timings were used for each participant by session. The same task settings were used across sessions as well, though different event timings were used to prevent participants from learning specific sequences.

Alternating Testing and Training Blocks Experiment. Six individuals (1 male, all right-hand dominant, mean age of 23.2, age range of 21-29) were randomized into two groups, and then each completed a single session of the AF-MATB. The data from two additional participants could not be used due to technical difficulties with the computer. Each session contained a total of 15 blocks of two minutes each. Blocks alternated between testing at medium intensity (8 blocks) and training at either low or high intensity (7 blocks), with training intensity set by group (Table 3).

The metrics that showed either significant or marginally significant training gains (COMM accuracy) were used to construct an overall performance measure that was sensitive to training improvements. Each metric's performance scores across blocks were converted into z-scores. This conversion was done separately for each subject, and scales were flipped if necessary so that positive z-scores indicated better performance. The converted metrics were then summed (with equal weighting by task) and the results normalized again. Mathematically, the conversion was RMAN-COMM z-scores=z(−2*z(RMAN deviation)+−1*z(COMM reaction time)+1*z(COMM accuracy)).

Calibration experiment. Five individuals (4 males, all right handed, mean age of 24, age range of 21-24) took part in a training MATB simulator with a total of 17 blocks, each lasting two minutes. Every other block including the first block (testing blocks) were set at a constant medium task intensity to collect performance values from which the RMAN-COMM z-scores could be calculated (see above). The training intensity for the blocks administered in between the testing blocks alternated amongst low, high, and medium. For analytical purposes, numeric values of one, two and three were assigned to the low, medium and high intensity conditions for these training blocks. The performance improvement associated with each training block was defined as the difference in performance for the testing blocks before and after the training block in question. A subject's profile (cognitive response function) represented the performance improvement during each training block as a function of the performance of the immediately preceding testing block and the training block's intensity. A visual representation of each profile's phenotypic surface was plotted using MATLAB R2017a (MathWorks Inc.).

Statistical Analysis: The R-squared values for the profiles (n=3) were calculated using regression analysis features in MATLAB R2018a (MathWorks Inc.). All other analyses were performed in RStudio version 1.0.136 (R Foundation for Statistical Computing) running R version 3.2.4 and MATLAB R2018a. The nominal alpha criterion level for all tests was set at 0.05, and correction for multiple comparison was achieved through p-value adjustment according to the False Discovery Rate (FDR) procedure.

### Example 2

From this proof of concept analysis of the two sets of parameters, three volunteers (D1, D2, D3) underwent a 62-minute session on the AF-MATB. The MATB involves using a joystick, keyboard and trackball to perform four different tasks simultaneously. The four tasks (TRCK, COMM, RMAN, SYSM) were blocked together with same difficulties, such as Medium difficulty for all four tasks, like the conditions in the Advanced Therapeutics publication, and dynamically changed throughout the session. The volunteers z-scores (performance scores) can be seen increasing and then decreasing over time. Two CURATE.AI N-of-1 profiles were calibrated per participant using the initial and last nine testing blocks (FIGS. 11-13). CURATE.AI profile surfaces changed over time, potentially reflecting each individual's change in performance over the duration of the session. These profiles further emphasized: 1) the inter-subject variability in performance improvement to training intensity and duration, 2) the dynamic nature of the CURATE.AI profile in capturing the individual's performance yielding different optimised training intensities throughout the duration of the session.

For the session to session analysis, both volunteer participants P6 and P3 underwent MATB training with two out of the four modules in MATB having the difficulty dynamically changed throughout each session, SYSM and RMAN. Both P6 and P3 began with concave behavior in their first session CURATE.AI profiles, with a shift in behavior being different in both participants beginning from session 3 and their CURATE.AI profiles becoming more and more different between both participants over the remaining sessions. Though more volunteer data and studies are needed, the preliminary analysis of the two participants seems to indicate that outcomes may be correlated to changes in CURATE.AI profiles and volunteer/patient responses, relating to being able to identify individuals to subpopulations (e.g. those that can improve the most, those that improve moderately, those that do not improve) and serve as a predictor of outcomes (e.g. z-score, training).

For volunteer P6 (FIG. 15), the MATB and CURATE.AI modulated intensities for calibration were run on desktop computer. From the first session, the CURATE.AI calibrated profile for P6 demonstrates concave behavior with most improvement in z-score identified from high intensity level of the SYSM module. The CURATE.AI calibrated profiles over the 7 sessions change dynamically from concave to saddle-like/convex, with the medium intensity level of RMAN module being identified for the most improvement in z-score.

For volunteer P3 (FIG. 16), the MATB and CURATE.AI modulated intensities for calibration were run on touchscreen laptop (equivalent to the handling of a tablet). From the first session, the CURATE.AI calibrated profile for P3 demonstrates concave behavior with most improvement in z-score identified from high intensity level of the SYSM module. The CURATE.AI calibrated profiles over the 8 sessions change dynamically from concave, saddle-like, convex, and finally concave again, with the high intensity levels of RMAN and SYSM modules being identified for the most improvement in z-score.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

What is claimed is:

1. A computer-implemented cognitive training process, comprising:
constructing a cognitive response profile for a user, the cognitive response profile representing a unique relationship between training intensity and performance for the user, thereby being a measurement of cognitive state or change in cognitive state as a function of one or more variables, by:
taking user-specific calibration measurements for the user, using sensors, using a sensor, over alternating training and testing blocks; and
obtaining a non-linear cognitive response function for the user based on the calibration measurements, using a calibration module, the non-linear cognitive response function representing a cognitive state, or change in cognitive state, as a function of the one or more variables, the one or more variables including one or more stimulus parameters of a stimulus to which the user is exposed, one or more current or past cognitive state values of the user to functionally define cognitive state or change in cognitive states from the past cognitive state or past cognitive states and a nature and/or intensity of the stimulus to which the user is exposed, and one or more variables that characterize an environment of the user, including one or more of temperature, background noise levels and lighting conditions;
initializing first values of one or more variables;
exposing the user to a first stimulus through a user interface, the first stimulus being characterized by respective the first values of the one or more variables, the user interface comprising one or more of a brain-computer interface, a speaker, a tactile feedback device and a display of a computing device, the first stimulus comprising a first prompt to perform multiple different tasks, the first prompt comprising one or more of an visible cue and an audio cue;
using a training module to determine, based on sensor measurements from at least one of a plurality of sensors, the sensor measurements being obtained across the one or more tasks from an interaction by the user with at least one sensor from the plurality of sensors, the at least one sensor including a camera for capturing images for determining cognitive performance measurements, one or more first cognitive performance values indicative of a response to the first stimulus, the response comprising at least one of a speed and an accuracy of the interaction, the interaction being characterised by interaction with an electromechanical sensor of a user input device, the user input device being one more or of a mechanical mouse, an optical mouse, a wireless keyboard, a hardwired keyboard, and a gesture-based input device;
using the training module to determine, using the non-linear cognitive response function, respective second values of the one or more stimulus parameters that will result in at least one improved cognitive performance value relative to the first cognitive performance value or values, the second values determined to optimize the cognitive response function given the one or more first cognitive performance values, based on a definition of the cognitive response function given by:
$$F ( x , p )$$
where x is the first (current) cognitive state value and p is the set of parameters characterizing a stimulus, wherein Fis optimised for fixed x to determine the second stimulus parameters as an optimum p that achieves optimised F, and where:
$$F ( x , p ) = F ( {S}^{′} , C ) = F ( {S}^{′} ) + {x}_{0} + \sum {x}_{i} {c}_{i} + \sum {y}_{ii} {c}_{i}^{2} + \sum {z}_{ij} {c}_{i} {c}_{j} + high - order terms$$
where S represents at least one of optimized cognitive, learning and training network mechanisms for the user and S′ represents, for the user, at least one of aberrant and sub-optimal, and/or at least one of average baseline cognitive, learning, training network mechanisms, xᵢ is the user's response coefficient to a factor i at amplitude/level cᵢ, and zᵢⱼ is the user's response coefficient to the interaction of manipulation i and manipulation j at respective amplitudes or levels of i and j, wherein each factor may be a stimulus parameter or a characteristic of the user;
exposing the user to a second stimulus through the user interface, the user interface comprising said one or more of the brain-computer interface, speaker, tactile feedback device and display of the computing device, the second stimulus comprising a second prompt to perform multiple different tasks, the second prompt comprising one or more of an visible cue and an audio cue, the second stimulus being characterized by the respective second values of the one or more stimulus parameters as measured by the sensors;
using the training module to obtain an updated non-linear cognitive response function based on the one or more first cognitive performance values, and/or based on one or more second cognitive performance values, the one or more second cognitive performance values indicative of a response to the second stimulus, the response being obtained from at least one of a speed and accuracy of the user interacting with at least one of the plurality of sensors in response to the second stimulus;
monitoring changes in the non-linear cognitive response function within a training session and/or across training sessions, using one or more processors;
outputting, to the user interface, an improvement in cognitive performance of the user in the response to the second stimulus when compared with the response to the first stimulus; and
classifying the user into a subpopulation based on similarity of the cognitive response function or changes in the non-linear cognitive response function to an average cognitive response function of the subpopulation.

2. A computer-implemented cognitive training process according to claim 1, wherein obtaining the cognitive response function comprises:
exposing the user to a plurality of stimuli, said plurality of stimuli being characterized by time-varying values of said one or more stimulus parameters; and
measuring, by at least one sensor, response data indicative of respective cognitive performance values corresponding to said time-varying values.

3. A computer-implemented cognitive training process according to claim 2, wherein at least two stimulus parameters are varied independently.

4. A computer-implemented cognitive training process according to claim 2, comprising fitting said response data to a functional form that is non-linear in the one or more stimulus parameters.

5. A computer-implemented cognitive training process according to claim 4, wherein the cognitive response function is a quadratic function of the one or more stimulus parameters.

6. A computer-implemented cognitive training process according to claim 1, further comprising making a prediction of a cognitive performance of the user based on the subpopulation.

7. A computer-implemented method according to claim 6, further comprising switching the cognitive training process if the prediction is that cognitive performance will not improve after further training sessions.

8. A computer-implemented method according to claim 6, further presenting stimuli to the user based on stimuli presented to the subpopulation that resulted in improvement in the cognitive response function of the subpopulation.

9. A computer-implemented cognitive training process according to claim 1, comprising classifying the user into a subpopulation of users; wherein the classification is based on one or more of: the cognitive response function; the updated cognitive response function; and the changes in the cognitive response function.

10. A computer-implemented cognitive training process according to claim 1, wherein one or more of said stimuli is presented via a user interface of a computing device.

11. A computer-implemented cognitive training process according to claim 1, wherein one of said one or more stimulus parameters is indicative of a training intensity.

12. A computer-implemented cognitive training process according to claim 10, wherein one or more of said stimuli comprises a prompt to provide an input at said computing device.

13. A computer-implemented cognitive training process according to claim 12, wherein the prompt is a prompt to provide an input at the user interface of the computing device.

14. A computer-implemented cognitive training process according to claim 1, wherein the at least one sensor is a sensor of a user input device.

15. A computer-implemented cognitive training process according to claim 1, wherein the cognitive response function is also a function of one or more previously measured cognitive performance values for the user.

16. A cognitive training platform, comprising:
at least one processor; and
at least one sensor in communication with the at least one processor and configured to obtain response data from a user;
wherein the at least one processor is configured to perform a cognitive training process according to claim 1.

17. A computer-implemented cognitive training process, comprising:
constructing a cognitive response profile for a user, the cognitive response profile representing a unique relationship between training intensity and performance for the user, thereby being a measurement of cognitive state or change in cognitive state as a function of one or more variables, by:
taking user-specific calibration measurements for the user, using sensors, using a sensor, over alternating training and testing blocks; and
obtaining a non-linear cognitive response function for the user based on the calibration measurements, using a calibration module, the non-linear cognitive response function representing a cognitive state, or change in cognitive state, as a function of the one or more variables, the one or more variables including one or more stimulus parameters of a stimulus to which the user is exposed, one or more current or past cognitive state values of the user to functionally define cognitive state or change in cognitive states from the past cognitive state or past cognitive states and a nature and/or intensity of the stimulus to which the user is exposed, and one or more variables that characterize an environment of the user, including one or more of temperature, background noise levels and lighting conditions;
initializing first values of one or more variables;
exposing the user to a first stimulus through a user interface, the first stimulus being characterized by respective the first values of the one or more variables, the user interface comprising one or more of a brain-computer interface, a speaker, a tactile feedback device and a display of a computing device, the first stimulus comprising a first prompt to perform multiple different tasks, the first prompt comprising one or more of an visible cue and an audio cue;
using a training module to determine, based on sensor measurements from at least one of a plurality of sensors, the sensor measurements being obtained across the one or more tasks from an interaction by the user with at least one sensor from the plurality of sensors, the at least one sensor including a camera for capturing images for determining cognitive performance measurements, one or more first cognitive performance values indicative of a response to the first stimulus, the response comprising at least one of a speed and an accuracy of the interaction, the interaction being characterised by interaction with an electromechanical sensor of a user input device, the user input device being one more or of a mechanical mouse, an optical mouse, a wireless keyboard, a hardwired keyboard, and a gesture-based input device;
using the training module to determine, using the non-linear cognitive response function, respective second values of the one or more stimulus parameters that will result in at least one improved cognitive performance value relative to the first cognitive performance value or values, the second values determined to optimize the cognitive response function given the one or more first cognitive performance values, based on a definition of the cognitive response function given by:
$$F ( x , p )$$
where x is the first (current) cognitive state value and p is the set of parameters characterizing a stimulus, wherein Fis optimised for fixed x to determine the second stimulus parameters as an optimum p that achieves optimised F, and where:
$$F ( x , p ) = F ( {S}^{′} , C ) = F ( {S}^{′} ) + {x}_{0} + \sum {x}_{i} {c}_{i} + \sum {y}_{ii} {c}_{i}^{2} + \sum {z}_{ij} {c}_{i} {c}_{j} + high - order terms$$
where S represents at least one of optimized cognitive, learning and training network mechanisms for the user and S′ represents, for the user, at least one of aberrant and sub-optimal, and/or at least one of average baseline cognitive, learning, training network mechanisms, xᵢ is the user's response coefficient to a factor i at amplitude/level cᵢ, and zᵢⱼ is the user's response coefficient to the interaction of manipulation i and manipulation j at respective amplitudes or levels of i and j, wherein each factor may be a stimulus parameter or a characteristic of the user;
exposing the user to a second stimulus through the user interface, the user interface comprising said one or more of the brain-computer interface, speaker, tactile feedback device and display of the computing device, the second stimulus comprising a second prompt to perform multiple different tasks, the second prompt comprising one or more of an visible cue and an audio cue, the second stimulus being characterized by the respective second values of the one or more stimulus parameters as measured by the sensors;
using the training module to obtain an updated non-linear cognitive response function based on the one or more first cognitive performance values, and/or based on one or more second cognitive performance values, the one or more second cognitive performance values indicative of a response to the second stimulus, the response being obtained from at least one of a speed and accuracy of the user interacting with at least one of the plurality of sensors in response to the second stimulus;
monitoring changes in the non-linear cognitive response function within a training session and/or across training sessions, using one or more processors;
outputting, to the user interface, an improvement in cognitive performance of the user in the response to the second stimulus when compared with the response to the first stimulus; and
classifying the user into a subpopulation based on similarity of the cognitive response function or changes in the non-linear cognitive response function to an average cognitive response function of the subpopulation.

18. A cognitive training platform, comprising:
at least one processor; and
at least one sensor in communication with the at least one processor and configured to obtain response data from a user;
wherein the at least one processor is configured to perform a cognitive training process including:
constructing a cognitive response profile for a user, the cognitive response profile representing a unique relationship between training intensity and performance for the user, thereby being a measurement of cognitive state or change in cognitive state as a function of one or more variables, by:
taking user-specific calibration measurements for the user, using sensors, using a sensor, over alternating training and testing blocks; and
obtaining a non-linear cognitive response function for the user based on the calibration measurements, using a calibration module, the non-linear cognitive response function representing a cognitive state, or change in cognitive state, as a function of the one or more variables, the one or more variables including one or more stimulus parameters of a stimulus to which the user is exposed, one or more current or past cognitive state values of the user to functionally define cognitive state or change in cognitive states from the past cognitive state or past cognitive states and a nature and/or intensity of the stimulus to which the user is exposed, and one or more variables that characterize an environment of the user, including one or more of temperature, background noise levels and lighting conditions;
initializing first values of one or more variables;
exposing the user to a first stimulus through a user interface, the first stimulus being characterized by respective the first values of the one or more variables, the user interface comprising one or more of a brain-computer interface, a speaker, a tactile feedback device and a display of a computing device, the first stimulus comprising a first prompt to perform multiple different tasks, the first prompt comprising one or more of an visible cue and an audio cue;
using a training module to determine, based on sensor measurements from at least one of a plurality of sensors, the sensor measurements being obtained across the one or more tasks from an interaction by the user with at least one sensor from the plurality of sensors, the at least one sensor including a camera for capturing images for determining cognitive performance measurements, one or more first cognitive performance values indicative of a response to the first stimulus, the response comprising at least one of a speed and an accuracy of the interaction, the interaction being characterised by interaction with an electromechanical sensor of a user input device, the user input device being one more or of a mechanical mouse, an optical mouse, a wireless keyboard, a hardwired keyboard, and a gesture-based input device;
using the training module to determine, using the non-linear cognitive response function, respective second values of the one or more stimulus parameters that will result in at least one improved cognitive performance value relative to the first cognitive performance value or values, the second values determined to optimize the cognitive response function given the one or more first cognitive performance values, based on a definition of the cognitive response function given by:
$$F ( x , p )$$
where x is the first (current) cognitive state value and p is the set of parameters characterizing a stimulus, wherein Fis optimised for fixed x to determine the second stimulus parameters as an optimum p that achieves optimised F, and where:
$$F ( x , p ) = F ( {S}^{′} , C ) = F ( {S}^{′} ) + {x}_{0} + \sum {x}_{i} {c}_{i} + \sum {y}_{ii} {c}_{i}^{2} + \sum {z}_{ij} {c}_{i} {c}_{j} + high - order terms$$
where S represents at least one of optimized cognitive, learning and training network mechanisms for the user and S′ represents, for the user, at least one of aberrant and sub-optimal, and/or at least one of average baseline cognitive, learning, training network mechanisms, xᵢ is the user's response coefficient to a factor i at amplitude/level cᵢ, and zᵢⱼ is the user's response coefficient to the interaction of manipulation i and manipulation j at respective amplitudes or levels of i and j, wherein each factor may be a stimulus parameter or a characteristic of the user;
exposing the user to a second stimulus through the user interface, the user interface comprising said one or more of the brain-computer interface, speaker, tactile feedback device and display of the computing device, the second stimulus comprising a second prompt to perform multiple different tasks, the second prompt comprising one or more of an visible cue and an audio cue, the second stimulus being characterized by the respective second values of the one or more stimulus parameters as measured by the sensors;
using the training module to obtain an updated non-linear cognitive response function based on the one or more first cognitive performance values, and/or based on one or more second cognitive performance values, the one or more second cognitive performance values indicative of a response to the second stimulus, the response being obtained from at least one of a speed and accuracy of the user interacting with at least one of the plurality of sensors in response to the second stimulus;
monitoring changes in the non-linear cognitive response function within a training session and/or across training sessions, using one or more processors;
outputting, to the user interface, an improvement in cognitive performance of the user in the response to the second stimulus when compared with the response to the first stimulus; and
classifying the user into a subpopulation based on similarity of the cognitive response function or changes in the non-linear cognitive response function to an average cognitive response function of the subpopulation.

19. The cognitive training platform of claim 18, wherein obtaining the cognitive response function comprises:
exposing the user to a plurality of stimuli, said plurality of stimuli being characterized by time-varying values of said one or more stimulus parameters;
measuring, by at least one sensor, response data indicative of respective cognitive performance values corresponding to said time-varying values;
wherein at least two stimulus parameters are varied independently;
further including fitting said cognitive response data to a functional form that is non-linear in the one or more stimulus parameters;
wherein the cognitive response function is a quadratic function of the one or more stimulus parameters; and
further comprising making a prediction of a cognitive performance of the user based on the subpopulation.
